(19) United States
(12) Patent Application Publication
(10) Pub. No.: US 2021/0353706 A1
(43) Pub. Date: Nov. 18, 2021

(54) INHIBITION OF ENDOGENOUS REVERSE TRANSCRIPTASE AND TARGETING OF CELLS FOR PROPHYLAXIS AND THERAPY OF CANCER AND AGING

(71) Applicant: Health Research, Inc., Buffalo, NY (US)

(72) Inventors: Andrei GUDKOV, East Aurora, NY (US); Katerina LEONOVA, East Aurora, NY (US)

(21) Appl. No.: 16/479,809

(22) PCT Filed: Jan. 23, 2018
(86) PCT No.: PCT/US18/14806

§ 371 Date: Jul. 22, 2019

Related U.S. Application Data
(60) Provisional application No. 62/449,380, filed on Jan. 23, 2017.

(51) Int. Cl. A61K 38/00; A61K 39/00; A61K 45/06; A61P 35/00

(52) U.S. Cl. CPC A61K 38/005; A61P 35/00; A61K 45/06; A61K 39/0011

(57) ABSTRACT

Provides are approaches for anticancer and antiaging treatments by administration of reverse transcriptase (RT) activity inhibitors that function to inhibit RT encoded by ORF2 of LINE1, or any RT that can participate in transcriptional activation of retroelements. Also provided are approaches to discovery of new compounds that can inhibit RTs, and identifying individuals who would benefit from treatment with such RT inhibitors, and treating such individuals. The methods are applicable for use in populations of cancer cells, pre-cancerous cells, somatic cells, and combinations thereof. Also provided are methods for monitoring the efficacy of a treatment that inhibits development of resistance to pharmaceutical agents, methods for prophylaxis and/or therapy for a pathology correlated with accumulation of somatic cells capable of spontaneously generating genetic alterations independently of cell divisions, wherein such cells express functional LINE1 elements. Also provides are methods for treating and/or preventing age-related conditions in an individual by administering an agent capable of selectively killing the cells that exhibit genetic instability as evidenced by expression of functional LINE 1. Also provided are methods for sensitizing cancer cells to a chemotherapeutic agent by administering an RT inhibitor.

### CROSS REFERENCE TO RELATED APPLICATIONS

This application claims priority to U.S. provisional application No. 62/449,380, filed Jan. 23, 2017, the disclosure of which is incorporated herein by reference.

### BACKGROUND

A major challenge of cancer treatment is tumor plasticity and adaptability resulting in acquisition of malignant properties and resistance to treatment. Cancer treatment has been traditionally focused on phenotypic differences between neoplastic and normal cells such as unconstrained growth, altered metabolism, increased angiogenesis, resistance to immune surveillance and metastatic capability. All tumor-specific traits arise due to the high level of genomic instability in tumor cells, which generates a near-endless number of altered variants for natural selection. Despite being such a fundamental driver of tumor development and progression, genomic instability has never been considered as a treatment target itself since it has always been viewed as a consequence of deficiencies in major pathways (DNA repair, replication control, mechanism restricting proliferation of damaged cells, etc.) without “druggable” targets. Thus, there is an ongoing and unmet need for a change in the approach to treating cancer and other undesirable outcomes that relate to genomic instability. The present disclosure is pertinent to this and other needs.

### SUMMARY

The present disclosure relates to a new approach for anticancer and antiaging treatments, an embodiment of which is summarized generally in FIG. 1. Embodiments of the disclosure comprise methods for prophylaxis and/or therapy for a variety of cancers, and other age-related conditions. The methods comprise administration of reverse transcriptase (RT) activity inhibitors in order to, among other effects, improve outcomes where conventional anti-cancer agents (that are not reverse transcriptase inhibitors) are inadequate, or are less effective than when an RT inhibitor is not administered. In embodiments, the RT inhibitors function to inhibit RT encoded by ORF2 of LINE1, or any RT that can participate in transcriptional activation of retroelements. The disclosure also includes approaches to discovery of new compounds, and identifying individuals who would benefit from treatment with such RT inhibitors to, for example, improve cancer and other therapeutic approaches by combining RT inhibitors with other pharmacological agents and/or other medical interventions, as further described below.

In one approach the disclosure provides a method for reducing genetic instability in a population of eukaryotic cells, wherein the method comprises introducing into the cells an RT inhibitor such that the activity of an RT encoded by ORF2 of LINE1 (wherein the RT is referred to as RT^{LINE}) in the cells is inhibited, and whereby the cells exhibit reduced genetic instability subsequent to the administration. Genetic instability includes but is not necessarily limited to chromosomal insertions, amplifications/deletions and point mutations that are driven at least in part by RT^{LINE}, which is not believed to be expressed in normal cells. Thus, the disclosure includes suppression of genomic instability in somatic cell populations by either inhibiting RT^{LINE} activity with drug agents, or stimulating the immune system to eradicate cells with activated functional LINE elements. Accordingly, the disclosure is pertinent to uses in populations of cancer cells, pre-cancerous cells, somatic cells, and combinations thereof. In certain aspects, the RT inhibitor can be introduced into progeny of the population of the cells, thereby obtaining progeny of the population of cells that comprise reduced genetic instability. In certain implementations, use of an RT inhibitor as described herein results in at least one of: fewer point mutations, fewer chromosomal insertions, fewer chromosomal deletions, fewer amplifications, a reduction in metastatic capability, a reduction in immortalization capacity, or a reduction in development to resistance to one or more pharmaceutical agents, wherein the reduction in genetic instability is relative to a control. Such effects can be realized in populations of cells. Any suitable control to measure such effects can be used, and such controls will be apparent to those skilled in the art given the benefit of this disclosure. In non-limiting embodiments, a control comprises a value for genetic instability obtained from cells in which activity of the RT is not inhibited.

In certain embodiments, the disclosure relates to suppression of point mutations that are caused at least in in part by endonuclease activity of an integrase component of the RT; and/or to insertions that comprise integration of new copies of one or more repeat elements, or integration of new pseudogenes, or a combination thereof; and/or to deletions that comprise loss of a segment of a chromosome that is surrounded by integrated copies of repeat elements; and/or to genetic amplifications that comprise new copies of genome fragments that contain one or more functional genes surrounded by newly integrated copies of repeat elements. In embodiments, the disclosure relates to RT administration to a population of cells such that at least some cells in the population of cells and/or progeny of said cells exhibit at least one of: i) less resistance to a pharmaceutical agent, and/or ii) reduced metastatic capability, or iii) less evidence of aging, relative to a control.

Methods of the disclosure can be performed for a population of cells that are maintained in vitro, or are present in a multi-cellular organism, and therefore include mammalian cells, including but not necessarily limited to human cells. In embodiments, the population of cells are comprised by a solid tumor, or are blood cancer cells.

In certain implementations, the population of cells is not infected by a human immunodeficiency virus (HIV), or other retrovirus, and the cells in the population do not comprise an integrated HIV provirus, or another integrated provirus of a retroviral origin.

In a non-limiting embodiment, the population of cells comprise cancer cells in an individual, and RT inhibitor administration is performed over a period of time during which: i) the cancer cells do not develop resistance to a chemotherapeutic agent that is also administered to the individual during the period of time, and/or ii) the cancer cells develop less resistance, relative to a control, to a chemotherapeutic agent that is also administered to the individual during the period of time.

In another non-limiting embodiment, the population of cells comprises somatic cells in an individual, and the administration of the RT inhibitor is performed over a period of time during which a frailty index of the individual is improved, or a worsening of a frailty index of the individual is slowed relative to a control.

In another aspect, the disclosure provides a method for characterizing whether or not a test agent(s) is a candidate for use as an RT inhibitor, the method comprising:

i) contacting cells that express an RT encoded by ORF2 of LINE1 (RT^{LINE}) with one or more test agents;

ii) testing for a change genetic instability in the cells; and

iii) determining that the test agent is a candidate by determining reduced genetic instability in the cells and/or their progeny relative to a control; or

iii) determining that the test agent is not a candidate by determining no reduction, or an increase, in genetic instability in the cells and/or their progeny, relative to a control. This aspect can further comprise contacting the cells with a chemotherapeutic agent to determine whether or not the RT inhibitor inhibits development of resistance to the chemotherapeutic agent. Such measurements can be made using any suitable approach, such as by measuring a change in genetic instability determined using a detectable reporter, wherein the detectable reporter is indicative of a DNA damage response.

In an aspect of the disclosure, a method of reducing/killing cells that exhibit genetic instability is provided. The method comprises administering to an individual in need thereof a composition comprising one or more agents that are capable of selectively killing the cells that exhibit the genetic instability, wherein the cells that exhibit the genetic instability can be identified by expression of functional LINE 1. In certain implementations of this approach, the one or more agents comprise all or a segment of one or two proteins encoded by LINE1 elements (ORF1 and ORF2), or the one or more agents comprise an expression vector(s) encoding the one or two proteins or the segment(s) thereof, wherein the protein(s) or the segment(s) thereof can stimulate an immune response against the cells that exhibit the genetic instability. In an embodiment, a composition administered to the individual further comprises one or more immunoadjuvants and/or immunomodulators, and/or the expression vector(s) encode the one or more immunoadjuvants and/or immunomodulators.

In any embodiment of this disclosure, the RT inhibitor can be a nucleoside analog RT inhibitor, or nucleotide analog RT inhibitor, or a combination thereof. Non-limiting examples of suitable RT inhibitors include stavudine and lamivudine, and additional examples of suitable RT inhibitors are described below.

In another aspect, the disclosure includes a method of identifying a stimulus that generates genetic instability. This approach comprises exposing eukaryotic cells to a test stimulus and testing the cells for an increase in activity of an RT encoded by ORF2 of LINE1 (RT^{LINE}), wherein an increase in in the activity of the RT indicates the stimulus generates the genetic instability.

In another aspect the disclosure provides a method for identifying an individual as a candidate for treatment with an RT inhibitor, wherein the RT is encoded by ORF2 of LINE1 (RT^{LINE}), and/or for treatment with a vaccine that stimulates expression of an immune response against cells that exhibit a genetic instability that can be identified by expression of functional LINE 1. These approaches comprise testing a sample from the individual to determine a measure of genetic instability, and comparing the measure of the genetic instability to a control, wherein more genetic instability relative to the control indicates the individual is a candidate for the treatment. In this and other embodiments of this disclosure, a measure of genetic instability comprises a value for at least one of: point mutations, chromosomal insertions, chromosomal deletions, gene amplifications, metastatic capability of cells from the individual, immortalization capacity of cells from the individual, expression of and/or presence of LINE1 DNA repeats in cells from the individual, and combinations of such measures. In one embodiment, the disclosure comprises identifying the individual as a candidate for treatment with an RT inhibitor, or receiving a test result with such an identification, and administering the inhibitor of the RT and/or the vaccine to the individual.

In another aspect, the disclosure includes a method for monitoring the efficacy of a treatment for inhibiting development of resistance to treatment with a pharmaceutical agent. This method comprises testing a sample of cells from an individual for a measure of genetic instability at a first time point during the treatment, and comparing the measure to a control, wherein a lack of increase in the genetic instability relative to the control indicates adequate efficacy of the treatment. Alternatively, an increase in the genetic instability at the first time point indicates inadequate efficacy of the treatment. Thus, the disclosure includes adjusting the dosage of the treatment and testing a second sample of cells from the individual for the measure of the genetic instability to determine if the dosage adjustment was adequate to inhibit development of the resistance. Such approaches are applicable to, for example, determining efficacy of a cancer treatment, wherein the agent is a chemotherapeutic agent or a vaccine.

In another embodiment, the disclosure provides a method for prophylaxis and/or therapy for a pathology correlated with accumulation of somatic cells capable of spontaneously generating genetic alterations independently of cell divisions, wherein such cells can be characterized by expression of functional LINE1 elements. This method comprises administering to the individual one or more RT inhibitors such that the activity of an RT encoded by ORF2 of LINE1 (RT^{LINE}) in the somatic cells is inhibited, and/or administering to the individual a vaccine such that an immunological response to the somatic cells is stimulated. In certain implementations of this approach, the pathology comprises cancer or other proliferation disorders, such as myelodysplastic syndrome or benign tumors, or aberrant immunomodulation, such as induction and/or exacerbation of inflammation that may initiate or accelerate aging and/or malignant transformation of the somatic cells. In embodiments, the pathology comprises spontaneous aging, and/or one or more conditions associated with aging and/or spontaneous aging. In embodiments, a change in the pathology can be detected by determining a change in frailty index for the individual. For example, a change in the frailty index can be measured at distinct time points to evaluate efficacy of the method.

In another aspect the disclosure includes a method for treating and/or preventing age-related conditions in an individual by administering to an individual in need thereof a composition comprising one or more agents that are capable of selectively killing the cells that exhibit genetic instability, wherein the cells that exhibit the genetic instability can be identified by expression of functional LINE 1. The one or more agents can comprise all or a segment of one or two proteins encoded by LINE1 elements (ORF1 and ORF2), or the one or more agents comprise an expression vector(s) encoding the one or two proteins or the segment(s) thereof, wherein the protein(s) or the segment(s) thereof can stimulate an immune response against the cells that exhibit the genetic instability. In embodiments, the composition comprising the one or more agents further comprises one or more immunoadjuvants and/or immunomodulators, and/or the expression vector(s) encode the one or more immunoadjuvants and/or immunomodulators, and may alternatively or further comprise nucleoside analog reverse-transcriptase inhibitors or nucleotide analog reverse-transcriptase inhibitors.

In another aspect, the disclosure provides a method of sensitizing cancer cells to a chemotherapeutic agent. This method comprises introducing into the cancer cells the chemotherapeutic agent and an RT inhibitor, wherein the cancer cells are not infected by HIV, or by another retrovirus, and wherein the cancer cells do not comprise an integrated HIV provirus, or another integrated provirus or retroviral origin.

### BRIEF DESCRIPTION OF THE FIGURES

FIG. 1 provides a schematic description of a new paradigm for anticancer and antiaging treatments based on pharmacological and immunotherapeutic targeting of cells expressing reverse transcriptase encoded by LINE1 family of elements (LINE1 RT).

FIG. 2 depicts a work-flow for a LINE1 retrotransposition assay as described in Moran et al. Cell 1996 87(5):917-27 (shown in box to the left). For the plate assays, HeLa cells were transfected with the reporter plasmid; appearance of neo-resistant colonies is indicative of retrotransposition events; the top plate images show stavudine is effective at concentrations above 5 mM. Lower panel demonstrates lack of effect of the indicated concentrations of stavudine on clonogenicity of HeLa cells.

FIG. 3 provides a graphical summary (left panel) and plate assays image (right panel) results obtained on from use of representative RT inhibitors stavudine (STV) and azidothymidine (AZT), and demonstrates suppress acquisition of drug resistant cell variants in a population of colorectal cancer cells HCT116.

FIG. 4 provides a graphical summary of results demonstrating that the representative HIV RT inhibitor stavudine prolongs tumor-free survival of p53-null C57BL6/j male mice.

FIG. 5 provides a graphical summary of results demonstrating the representative RT inhibitor stavudine delays occurrence and reduces incidence of tumor relapses following initial response to standard chemotherapy in mouse spontaneous neuroblastoma model.

### DESCRIPTION OF INVENTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

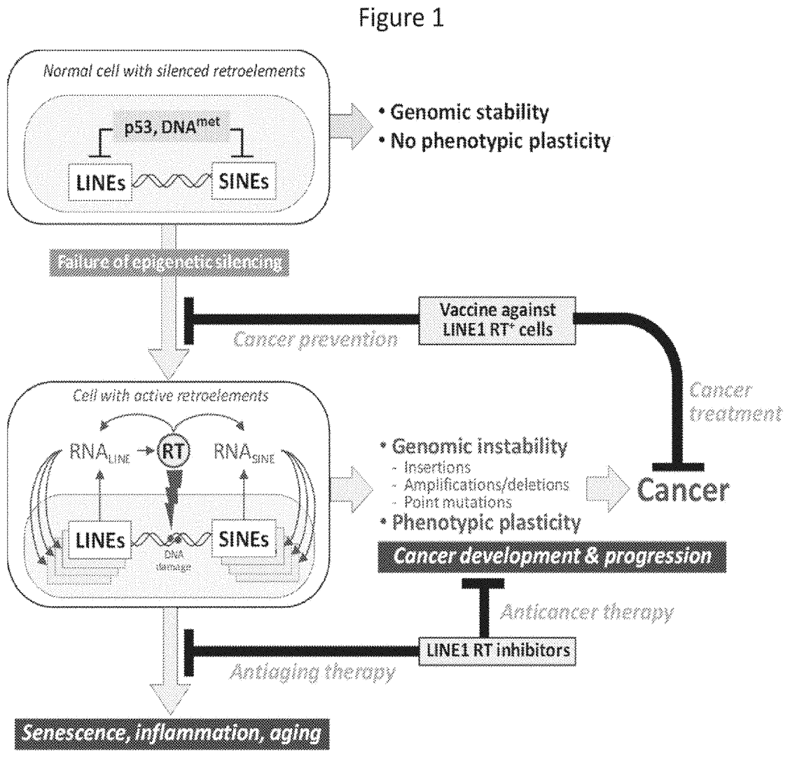

The present disclosure generally relates to the concept that transcriptional activation of retroelements can occur either spontaneously or following specific stresses (i.e., genotoxic conditions) in rare somatic cells of mammalian organisms. A non-limiting embodiment of this disclosure is presented schematically in FIG. 1, and is discussed further in the Examples. In more detail, and without intending to be constrained by any particular theory, it is considered this “desilencing” results in expression of endogenous reverse transcriptase (RT) encoded by LINE1 elements, which drives synthesis of cDNA copies of a variety of RNA (i.e., LINE1 RNA, RNA of SINE elements, protein-coding mRNAs, tRNAs, small nuclear RNAs, etc.) and integration into random sites of cellular DNA. This leads to insertional mutagenesis associated with gene inactivation or synthesis of aberrant products (integration into open reading frames), modulation of expression (integration into regulatory regions), gene amplification (when similar repeats are integrated around certain gene facilitating homologous recombination), etc. Moreover, RT of LINE1 generates DNA breaks via its endonuclease activity thereby inducing point mutations. This process results in a high degree of genomic instability in somatic cell populations leading to increased risk of cancer development and accumulation of cells with altered properties and affected normal functions thereby contributing to overall decline in functional performance of the organism observed with age. The disclosure is based in part on the approach that, because all these processes depend at least in part on the endogenous RT of LINE1 elements—they can be inhibited and/or prevented by using targeting the RT and/or cells that express this RT. Thus, in various embodiments, the disclosure relates to inhibition and/or prevention of cancer, aging and age-related diseases and for suppression of cancer progression. Accordingly, in embodiments the disclosure comprises methods for reducing the occurrence of genetically altered cells, such as cells with elevated genomic instability, phenotypic plasticity and adaptability, and other features that are described more fully herein. In embodiment, the disclosure pertains to reducing such genetically altered cells in a population of cells, such as cancer cells in general, methods of this disclosure comprise exposing such cells to one or more inhibitors of RT and/or vaccination against such cells.

The disclosure includes in certain approaches methods of inhibiting development of genetically altered variants from genetically altered cells in cancer cell populations, wherein the cells have previously acquired a high degree of genomic instability, such as cells that express functional LINE1 DNA repeats). The disclosure includes in certain aspects targeting genetically altered cells with RT inhibitors and/or vaccines or other immunomodulating agents that selectively target genetically altered cells.

In embodiments “genetically altered cells” include cells that acquired at least one of point mutations, insertions, deletions, and amplifications, due at least in part to the activity of RT. Genetically altered cells can include cells that have acquired novel processed pseudogenes, and cells that, because of their genetic modification, become resistant to cancer treatment or acquire other properties which confer selective advantages to such cells. Genetically altered cells include but are not necessarily limited to cells of melanoma, neuroblastoma or other cancers that acquired amplification of protooncogenes of, for example, MYC family genes, (i.e., CMYC, NMYC), and breast, ovarian or prostate cancer that have acquired amplified HER2/NEU genes, and cells of prostate cancer that have acquired amplification or point mutations in an androgen receptor gene, and thereby have become castration resistant, and other examples of treatment resistance cancer cells. Genetically altered cells include cells with chromosomal abnormalities that distinguish them from other cells in a population, including cells with chromotripsis, etc. In certain aspects, the disclosure pertains to cells that have developed resistance to one or more cancer treatments, which means the cells have the ability to continue proliferation in the presence of chemotherapeutic drug(s), targeted anticancer agent(s), ongoing immunotherapy or radiation treatment to which the cells have developed resistance. In embodiments of this disclosure, the development of such resistance is inhibited and/or prevented by administration of an RT-inhibiting agent as described herein.

Any inhibitor of reverse transcriptase can be used in methods of this disclosure. Thus, any pharmaceutical agent(s) that possess inhibitory activity against polymerase and/or endonuclease activities against reverse transcriptase encoded by LINE elements can be used. In embodiments, the RT is encoded by ORF2 of LINE1, or is encoded by ORF2 of LINE2 elements that are present in mammalian genomes. Suitable RT inhibitors include but are not limited to those RT inhibitors of HIV, which possess cross reactivity with RT encoded by ORF2 LINE1 (i.e., stavudine). Suitable agents also include inhibitors of integrase of HIV, and accordingly possess cross reactivity with endonuclease activity of RT encoded by ORF2 of LINE1. In certain approaches, the RT inhibitor can be a nucleoside analog or nucleotide analog RT inhibitor, or a non-nucleoside or non-nucleotide RT inhibitor. In embodiments, an RT inhibitor can comprise a compound or complex of compounds having dual inhibitory effects against RT and integrase. Thus, in various embodiments, disclosure includes administering to an individual in need thereof one or more compounds that function as Nucleoside analog reverse-transcriptase inhibitors (NARTIs or NRTIs), or Nucleotide analog reverse-transcriptase inhibitors (NtARTIs or NtRTIs). Non-limiting examples of such compounds include AZT (Zidovudine), Didanosine, Zalcitabine, Stavudine, Lamivudine, Abacavir, Emtricitabine, Entecavir, Tenofovir, Adefovir, zalcitabine (ddC), lamivudine (3TC), and emtricitabine (FTC), abacavir (ABC) and entecavir (ETV), didanosine (ddI), tenofovir (TDF), and adefovir (ADV). In certain embodiments, Non-nucleoside reverse-transcriptase inhibitors (NNRTIs) can be used, non-limiting examples of which include Efavirenz, Nevirapine, Delavirdine, Etravirine and Rilpivirine. In one embodiment, the disclosure includes administering Portmanteau inhibitor.

In addition to encompassing use of any known RT inhibitor, the disclosure includes methods of identification of new inhibitors of the endonuclease activity of RT LINE1 comprising screening candidate agents for their ability to inhibit RT, and/or for their ability to block DNA a damage response, such as in experimentally generated cells in culture carrying any type of a reporter indicative of DNA damage response (i.e., p53-responsive reporters).

In certain embodiments, the disclosure relates to pharmacological and immunotherapeutic approaches for inhibition and/or prevention of pathological consequences stemming from accumulation in normal somatic cell populations of mammalian body cell variants capable of spontaneously generating genetic alterations independently of cell divisions. A common property of such cells is expression of functional LINE1 elements. Such pathological consequences can comprise cancer, other proliferation disorders (i.e., myelodysplastic syndrome, benign tumors) and immunomodulation (i.e., induction of inflammation) that may contribute to aging and malignant transformation. The pathological consequences that are addressed by embodiments of this disclosure also include the naturally occurring frailty of elderly humans and non-human animals that result from spontaneous aging. Thus, the efficacy of treatment can be determined in one aspect by measurement of the frailty index of an individual. Determination of a frailty index (FI) can be performed according to known approaches. In certain embodiments, a frailty index can be calculated a number of parameters, including a ratio of a total number of deficits measured and assigned an FI score, such as an FI score between 0 (no deficits=fit) and 1 (all deficits present=frail). Therefore, in on example, a higher FI indicates poorer health of an organism, and/or the biological age of an organism. In general, and without intending to be bound by theory, it is considered that frailty is a state of increased vulnerability to adverse outcomes. An FI index of this disclosure can include measuring any suitable number of accumulated deficits, such as at between 5-30 deficits. In embodiments, a frailty index as described in Searle, et al., (2008) DOI: 10.1186/1471-2318-8-24, can be used, the disclosure of which is incorporated herein by reference. In embodiments, a frailty index is calculated as described in U.S. patent publication no. 20150285823, from which the description of frailty index, frailty index parameters, and frailty index determination is incorporated herein by reference. Frailty index parameters can include but are not limited to of weight, grip strength, blood pressure, complete blood count, and cytokine analysis.

Methods for administering compositions of this disclosure, including RT inhibitors and immunogenic compositions, can comprise parenteral, intraperitoneal, intrapulmonary, oral, mucosal and topical administrations. Parenteral infusions include intramuscular, intravenous, intraarterial, intraperitoneal, and subcutaneous administration. The amount of the RT inhibitor and/or immunogenic agent (or polynucleotide encoding it) and any other active agent to be included in a composition and/or to be used in the method can be determined by those skilled in the art, given the benefit of the present disclosure. Thus, in one embodiment, an effective amount of a composition of the invention is administered. An effective amount can be an amount that that alleviates disease symptoms associated with cancer, aging, and/or age-related diseases. For immunogenic compositions, an effective amount is such that as a consequence of vaccination at least some cells that express RT and/or comprise genetic instability as described herein are eliminated from an individual. Effective amounts of RT inhibitors are those that inhibit RT function to reduce or eliminate creation of genetic instability that is described herein.

In certain embodiments, a composition comprising a vaccine and/or an RT inhibitor is administered to an individual in need thereof. The individual can be diagnosed with, suspected of having, or be at risk for any cancer, and/or for any age-related condition. In certain implementations the disclosure is for prophylaxis and/or therapy for age-related diseases such as Alzheimer's disease, type II diabetes, macular degeneration, chronic inflammation-based pathologies (e.g., arthritis), and/or to prevent development of cancer types known to be associated with aging (e.g., prostate cancer, melanoma, lung cancer, colon cancer, etc.), and/or with the purpose of improving the outcome of cancer treatment by radiation or chemotherapy. In certain embodiments, the individual treated using an approach of this disclosure is selected based at least in part on having cells comprising genetic instability, wherein the genetic instability is as further described herein. In embodiments the individual does not have, and/or has not contracted a retroviral infection in embodiments, the individual is not HIV+ and has not been diagnosed as HIV+. In embodiments, the individual is characterized as having low risk of contracting an HIV infection, or another retroviral infection. In embodiments the individual is selected for treatment according to the present invention based on a determination of a frailty index, and/or by determining one or more parameters described herein that indicate genomic instability. In embodiments, the individual has not received an RT inhibitor before a first treatment with an RT inhibitor as described herein.

An effective amount of a composition administered according to this disclosure can vary depending on pharmaceutical formulation methods, administration methods, the patient's actual or calculated age, body weight, sex, overall health, type and stage of cancer if cancer is being treated, diet, administration time, administration route, and other factors that will be apparent to those skilled in the art. Compositions can be administered once, or over a series of administrations, and may be administered chronically so as to maintain genomic stability, and/or to improve genomic stability. Thus, in embodiments the disclosure comprises administering RT inhibitor(s) over an extended period of time, and thus can include chronic administration of effective doses of pharmacological agents capable of suppressing the polymerase and/or endonuclease function of RT LINE1. Immunotherapeutic methods as described above comprise promoting selective killing of cells that express functional LINE 1, and thus include vaccination approaches. Vaccines of this disclosure can comprise compositions that include one or both proteins encoded by LINE1 elements (ORF1 and ORF2), and can include immunogenic fragments thereof. The proteins can be combined with any suitable immunoadjuvant(s), including but not limited to alum and/or agonists of innate immunity receptors, and may be provided in association with such compositions, including non-covalent and covalent associations. In another approach, stimulation of an immune response can be achieved using any suitable recombinant vector(s) encoding one or both proteins of LINE11, and/or immunogenic fragments thereof. Suitable recombinant vectors include but are not limited to adenoviral, lentiviral and adenoassociated viral vectors. Such vectors can also express immunomodulators (i.e., bacterial flagellin) as immunoadjuvants. In certain embodiments, cells that are targeted and reduced or eliminated from somatic cell populations by a stimulated immune system according to this disclosure include cells that express LINE1, or cells that have acquired novel processed pseudogenes or have chromosomal abnormalities.

In more detail, and without intending to be bound by any particular theory, it is considered that despite being such a fundamental driver of tumor development and progression, genomic instability has not previously been considered as a treatment target itself since it has been viewed as a consequence of deficiencies in major pathways (DNA repair, replication control, mechanism restricting proliferation of damaged cells, etc.) without “druggable” targets. The present disclosure introduces an alternative paradigm based on the interpretation that the genomic instability of tumor cells is caused by an active process involving desilencing and reverse transcriptase-driven amplification of repetitive retroelements that comprise (normally in silent form) nearly half of the mammalian genome. This process is driven by endogenous reverse transcriptase (RT) encoded by rare copies of repeats belonging to the LINE family (RT^{LINE}) which retain their functionality. The disclosure relates in part to the observation that transcriptional desilencing of retroelements frequently occurs in cancer and can be provoked by loss of p53, which normally acts as an epigenetic repressor of the “DNA repeatome”. Furthermore, tumors frequently acquire activation of endogenous RT^{LINE} expression that is not observed in normal cells besides early embryogenesis and neonatal brain. Thus, tumors frequently acquire all of the necessary components for a “genomic instability generator” (RT as well as RNA templates for amplification) that may produce three types of mutations: insertions (integration of new copies of repeats and new processed pseudogenes), amplifications/deletions (provoked by homologous recombination between newly integrated copies of repeats) and point mutations (via endonuclease activity of the integrase of RT^{LINE}). Since all of these events are considered to be driven by a single enzyme —RT^{LINE}—that is not expressed in normal cells, the disclosure relates to suppression of genomic instability in somatic cell populations by either inhibiting RT^{LINE} activity with drugs or stimulating the immune system to eradicate cells with activated functional LINE elements. In connection with this, we demonstrate that stavudine, a drug used for HIV treatment and capable of inhibiting RT^{LINE} activity, greatly reduced the frequency of gene amplification in vitro and significantly extended the cancer-free lifespan of tumor-prone p53-deficient mice. Thus, the present disclosure pertains to the interpretation that: (i) activation of retroelements driven by RT^{LINE} is a major contributor to genomic instability underlying cancer development and progression, and (ii) eradication of cells with activated RT^{LINE} through immunotherapy (vaccination) can be used for cancer prevention, and well as other features that will be apparent to those skilled in the art from this disclosure. In certain aspects, the disclosure includes vaccines against antigens of LINE1-expressing cells which are tested for prevention of engraftment, and treatment of transplanted tumors expressing mouse LINE1. Successful vaccines can demonstrate cancer preventive efficacy in four models of cancer-prone mice genetically predisposed to breast (MMTV-neu), prostate (PTEN hemizygous) or multiple cancers (p53 deficient, p53 hemizygous+irradiation).

As discussed above, in certain embodiments the disclosure comprises targeting cells that comprise/express DNA retroelements. These elements, namely short and long interspersed nuclear elements (SINEs and LINEs), together comprise nearly 40% of most mammalian genomes. More than 65 million years ago, all major classes of SINEs and LINEs massively invaded the genomes of predecessors of mammals via a mechanism resembling naturally occurring PCR driven by reverse transcriptase (RT). The source of RT enzyme is provided by LINE1 elements ancient primitive RNA viruses that have only two open reading frames (ORFs), one of which, ORF2, encodes RT. Massive amplification of SINEs and LINEs coincided in evolution with emergence of all major archetypes of mammals, presumably providing natural selection with enormous genetic and phenotypic diversity due to insertional mutagenesis. In the genomes of current mammals, amplification of SINEs and LINEs is effectively blocked as they are transcriptionally silent making neither RT, nor RNA templates available for retrotranspositions. We have shown that epigenetic silencing of retroelements is controlled cooperatively by p53 and DNA methylation. If both fail, massive transcription of virus-like RNAs induces a suicidal interferon response (Leonova, K. I., et al. p53 cooperates with DNA methylation and a suicidal interferon response to maintain epigenetic silencing of repeats and noncoding RNAs. Proc Natl Acad Sci USA 110, E89-98 (2013)). Nevertheless, all these safeguards cannot completely prevent activity of retroelements as evident from the analysis of tumors that frequently acquire expression of RT^{LINE} by a few remaining functional elements among the ˜100,000 copies of LINEs present in the human or mouse genome. Tumors also commonly activate transcription of virus-like RNAs, thus providing RNA templates for RT^{LINE} to initiate the process of amplification of retroelements. Consistently, appearance of newly integrated copies of both SINEs and LINEs has been demonstrated in many tumors and it has been shown that scale of RT activation correlates with stages of tumor progression.

Without intending to be limited to any particular model, the present disclosure is based in part on the interpretation retroelement activity is a major source of genomic instability leading to cancer development and progression an is part of an active ongoing process which, unlike other mechanisms involved in genomic instability (e.g., DNA replication inaccuracy, DNA repair inefficiency, etc.), is potentially druggable in this regard, amplification of retroelements is believed to be completely dependent on the only known endogenous source of RT encoded by a few highly homologous copies of LINE1s. The present disclosure demonstrates that small molecule inhibitors of endogenous RT^{LINE} create a decrease in the frequency of gene amplification in tumor cells in vitro and a delay in spontaneous tumor development in p53-deficient mice. Thus, this disclosure indicates that RT activity can substantially affect genomic stability and its inhibition can reduce cancer development and progression. Hence, this disclosure presents a paradigm-shifting strategy to cancer prevention, as well as addressing aging and age-related diseases, in which the plasticity cancer and other cells is approached as a druggable trait, and may provide for significant improvement of the efficacy of current treatments by suppressing development of drug-resistant tumor cell variants, as well as preventing and/or inhibiting spontaneous aging and age-related diseases. Accordingly, for the first time, the present disclosure reveals that genomic instability—the major property of cancer that makes it undefeatable—is a trait amenable to pharmacological and immunotherapeutic (vaccines) targeting. While there are published reports on anticancer activity of RT inhibition, they are all based on an assumption that RT is essential for tumor cell viability. In contrast, in certain embodiments, the present disclosure relates to the concept of not targeting tumor viability (and other cells involved in aging and age-related disorders), but rather their “creativity”—genetic and phenotypic plasticity. The disclosure, therefore, defines a new role for RT inhibitors in oncology and aging as drugs for prevention of cancer development and progression, including acquisition of treatment-resistance, and related approaches and outcomes in the area of aging and age-related conditions. Further, in addition to its conceptual novelty, the present disclosure includes a number of important technical innovations. For example, the disclosure includes a retroposition-dependent suicidal element suitable for both in vitro use and in transgenic mice. Also included is a series of new rationally designed recombinant proteins for effective anti-LINE vaccination. Vectors, genetically modified cell lines and transgenic mice resulting from this disclosure (i.e., cell lines and mice with active RTs encoded by different constructs, molecular vaccines, etc.) will form a new tool set useful for studies of the biological role of retroelements in evolution and in cancer prevention, predisposition and progression studies. All of these embodiments are encompassed by this disclosure.

It will be recognized from the foregoing that the disclosure is based at least in part on the interpretation that LINE RT-driven amplification of retroelements is a major driver of genomic instability that contributes to both cancer origin and progression (acquisition of new properties such as drug resistance, metastatic capacity, etc.). The disclosure also includes recognition of the influence of genomic instability on aging and age-related disorders. Our interpretation, which is not intended to be limiting, is supported by: (i) the proven connection between explosive repeat amplification and emergence of all major morphological archetypes in mammalian evolution, (ii) the role of p53 in epigenetic silencing of retroelements (and their frequent desilencing in p53-deficient cells), (iii) documented desilencing and amplification of repeats in tumors, and (iv) data from our studies testing LINE RT inhibition in vitro and in vivo.

As discussed above, the present disclosure reveals cancer's phenomenal plasticity and ability to evade natural anticancer mechanisms and applied treatments as a “druggable” active process driven by a single endogenous enzyme—RT^{LINE}. RT-dependent genomic instability likely involves three types of events: (1) insertional mutagenesis caused by integration of new copies of LINEs and their RT-driven SINEs into functional areas of the genome, (2) deletions and amplifications provoked by homologous recombination between newly integrated repeats, and (3) point mutations triggered by the activity of LINE1 RT-associated endonuclease. The current disclosure includes evaluating the effect of enforced expression of RT^{LINE} in different contexts on genomic instability of somatic cells and to determine the contribution of each type of genome-destabilizing event in the overall mutagenic effect of RT^{LINE} and to address specific mechanistic questions regarding the role of RT origin, contribution of ORF1, etc.

Among ˜100,000 “dead” LINE1 elements (with ORFs interrupted by numerous mutations), there remain only a few functional highly homologous copies of LINE1s in the human genome. Except for a short period during early embryonic development and brain maturation after the birth, they are deeply silenced at the epigenetic level in normal somatic cells, but frequently become transcriptionally and translationally active in tumors. The present disclosure includes the interpretation that this contributes to the genomic instability of tumors, thus highlighting RT^{LINE} as a target antigen for cancer immunotherapy aimed at prevention of cancer development and progression. The disclosure thus includes development of a vaccine capable of eliminating cells with active LINE1s. The disclosure accordingly includes generating vaccines and determining their (i) immunotherapeutic efficacy in experimental models of tumors with active LINE1s, and (ii) immunoprophylactic efficacy in several models of cancer-prone mice. The disclosure also relates to use of RT-inhibitors and such vaccines in methods of inhibiting aging and/or age-related disorders.

The disclosure includes use of assays to quantitatively assess the frequency of (i) retrotransposition, (ii) spontaneous gene amplification, and (iii) various gene-inactivating mutations (point mutations, deletions, etc.). The first assay will utilize a recombinant vector named RTAD (for RetroTransposition Activity Detector) similar to the ORFEUS, a vector previously constructed to detect cells with ongoing retrotransposition. RTAD is made from a mouse functional LINE element with ORF2 replaced by a reporter cassette consisting of puromycin-resistance and herpesvirus TK (ganciclovir sensitivity) genes under the ubiquitin promoter cloned in the orientation opposite to LINE1 transcription. In this reporter cassette, the promoter is inactivated by an intron. Thus, puro and HSV TK expression is only possible if the intron is spliced out and a functional cDNA copy is synthesized from the spliced RNA. Since that latter step requires reverse transcription, RTAD serves as a detector of RT activity. Puromycin resistance will provide an indication of the frequency of cells in a population with functional RT. Ganciclovir selection can then be used to eradicate such cells and assess the impact of RT activity on genomic instability.

Gene amplification can be assessed by measuring the frequency of occurrence of cell variants resistant to N-phosphonoacetyl-L-aspartate (PALA), a small molecule inhibitor of aspartate transcarbamylase of CAD protein essential for UMP biosynthesis. In human cells, PALA resistance is associated almost solely with CAD overexpression due to amplification of the CAD gene. Therefore, this can serve as a quantitative indicator of gene amplification. Usually undetectable in normal cells, gene amplification frequency can be as high as 10-5 in tumor cells. The finding that p53 inactivation and DNA demethylation by decitabine both greatly increase the frequency of gene amplification support the possibility of involvement of retroelements (which are inhibited at the epigenetic level by p53 and DNA methylation) in this process. Consistent with this, we found that cultivation of tumor cells in the presence of non-toxic concentrations of RT inhibitors stavudine (STV) or azido cytidine (AZT), both known to be effective against RTLINE1, greatly decreased the incidence of PALA-resistant colonies.

The frequency of gene-inactivating mutations can be determined using another series of detector cell lines transduced with lentiviral construct PURTK expressing the above-described puro-HSV TK reporter cassette conferring puromycin resistance and ganciclovir sensitivity. The frequency of occurrence of ganciclovir-resistant clones in a cell population is a quantitative indicator of HSV TK gene inactivation by mutations. Therefore, these cells can be used to assess the effect of different factors on spontaneous mutagenicity.

Two types of RT^{LINE1} activity detector tools (with RTAD and PURTK) can be generated from the same sets of mouse tumor-derived cell lines. These tools can be used to assess the dependence of three types of genomic instability —insertional mutagenesis, gene amplification and point mutations—on the activity of RT from different sources: (i) full-length LINE (natural and strong promoter-enforced ORF1 and ORF2 coexpression), (ii) RT^{LINE1} alone (to determine the impact of ORF1), and (iii) RT of different origin (MoMuLV RT—alone or within LINE1 in the place of ORF2). The nature of expected mutational events in selected cells can be analyzed as described herein.

A set of “modifier” constructs can be used to determine the ability of RT expression to provoke spontaneous transformation in vitro. Two model systems that have been successfully used in our previous work will be used, both involving conversion of mouse Balb/c 3T3-derived non-transformed fibroblasts (clones (12)1 and (10)1 into transformed anchorage-independent tumorigenic cells. (10)1 cells are p53-deficient and prone to transformation with activated mutant Ras while (12)1 cells are p53-wt and require p53 inactivation for Ras-mediated transformation. Both can be transfected with all modifier constructs either alone (line (12)1) or in combination with V-Ha-Ras under the SV40 promoter (line (12)1). Frequencies of cells in transduced populations capable of anchorage-independent growth due to acquisition of mutations either activating dominant oncogenes or suppressing p53 (in the case of (10)1 and (12)1, respectively) can be measured.

It is expected that RTAD will provide a powerful new genetic tool not only for monitoring the activity of retroelements, but also for controlling genomic stability in cell populations by eradicating cells with activated repeats. Thus, the disclosure includes creating transgenic mice carrying RTAD in their germline for use as a model for in vivo assessment of the feasibility of eradicating RT^{LINE1}-expressing cells for cancer prevention, as well as aging and age-related disorders.

As discussed above, the disclosure encompasses approaches to induction of adaptive immunity against cells with active retroelements and to assess their efficacy for cancer prophylaxis and therapy. The disclosure encompasses all vaccines that can function in methods of the disclosure. In certain embodiments, the disclosure provides two different types of vaccines: (i) protein-based (split vaccine) and (ii) gene-based adenoviral vaccine, and combinations of such approaches for use in, for example, prime-boost regimens. The disclosure includes structural optimization of LINE1's ORF1 and ORF2 which can include removal of immunosuppressive T-reg epitopes and adjusting codon contents to improve antigens' expression levels. Genetic sequences encoding secreted forms of ORF1 and ORF2 can be created by, for example, addition of leader peptides according to established approaches. In addition to recombinant ORF1 and ORF2 antigens, various combinations of molecular adjuvants representing agonists of pattern-recognition receptors (TLR7/8, NOD1/2) can be included in split vaccine. Immunogenicity of developed gene-based vaccines can be increased by addition into the adenoviral vector additional ORF encoding entolimod—a derivative of salmonella flagellin, an agonist of TLR5 and powerful immunoadjuvant. Vaccine formulations can be tested for their ability to activate cellular components of immunity, such as by FACS-based detection of lymphocyte activation markers (CD107, CD69, CD25, CD44), analysis of proliferative response and cytokine production (IFN-γ, IL-2, TNF) of antigen-specific CD4+ and CD8+T lymphocytes upon their stimulation with ORF1 and ORF2 antigens. Anti-tumor efficacy of both gene-based and split vaccine formulations can be tested using any suitable approaches, including but not limited to in vivo tumor models that include genetically modified tumor cells described above, and expressing LINE1's ORF1/ORF2 antigens along with RTAD and grown as s.c. transplanted tumors in syngeneic mice. Functional efficacy of experimental vaccines in transplanted models can be tested in prophylactic and treatment modes as described elsewhere, such as for entolimod-based anticancer vaccines. Efficacy of anti-LINE1 vaccines for prophylaxis of spontaneous cancers can be tested in established genetic models of mice predisposed to breast (MMTV-neu), prostate (PTEN hemizygous) or multiple cancers (p53 deficient, p53 hemizygous with total body irradiation). Animals can be immunized with vaccine preparations around 12 weeks of age and monitored for cancer development along with placebo control groups. Animals will be reserved for intermediate analysis at times of detectable hyperplastic lesions in target organs. Implementation of these approaches is expected to demonstrate substantial prophylactic and treatment effects of anti-LINE1 vaccines (extension of tumor-free life, tumor growth suppression or regression). Expression of LINE1 antigens can be compared in tumors growing in vaccinated and control animals.

In view of the foregoing description, it will be apparent that this disclosure is expected to lead to numerous practical medical applications and to provide broadly applicable solutions for treatment and prevention of cancer, aging and age-related diseases and conditions which currently have no suitable treatment approach or universal target.

The following Examples are intended to illustrate but not limit the invention.

### Example 1

FIG. 1 provides, without intending to be constrained by any particular theory, a schematic description of a new paradigm that sets forth new methods of anticancer and antiaging treatments that is based on pharmacological and immunotherapeutic targeting of cells expressing reverse transcriptase encoded by LINE1 family of elements (LINE1 RT). In particular, FIG. 1 schematically depicts cancer origin and progression, as well as aging, in mammals as a result of spontaneous epigenetic derepression of endogenous retrotransposons and their expansion in the genome via mechanism driven by LINE1 RT. LINE1 RT enables synthesis and integration of cDNA copies of a variety of cellular RNA including RNA of LINE1 and SINE retrotransposons. This process provokes different type of mutagenesis (insertional, deletions, amplifications, point mutations) and activation of inflammation (via DNA damage response and interferon induction). LINE1 RT-induced mutagenesis leads to accumulation of genetically and phenotypically altered cancer-initiating cells. If this process proceeds in a cancer cell population, it accelerates tumor progression involving acquisition of new malignant and treatment resistant properties. In populations of normal somatic cells, it leads to accumulation of cells with proinflammatory properties (e.g., senescent cells) and drives age-related increase in spontaneous sterile systemic inflammation. LINE1 RT inhibitors, including small molecules capable of inhibition of polymerase and/or endonuclease activity of this multifunctional enzyme, when applied to healthy organism, are expected to suppress aging and cancer development and can be used for cancer and aging prophylaxis. When applied to cancer-bearing organism, such inhibitors suppress cancer ability to accumulate additional mutations thereby slowing down cancer progression. As described above, the RT inhibitors are expected to be suitable for use combination with any cancer treatment to make the treatment more effective via suppression of acquisition of treatment resistance.

### Example 2

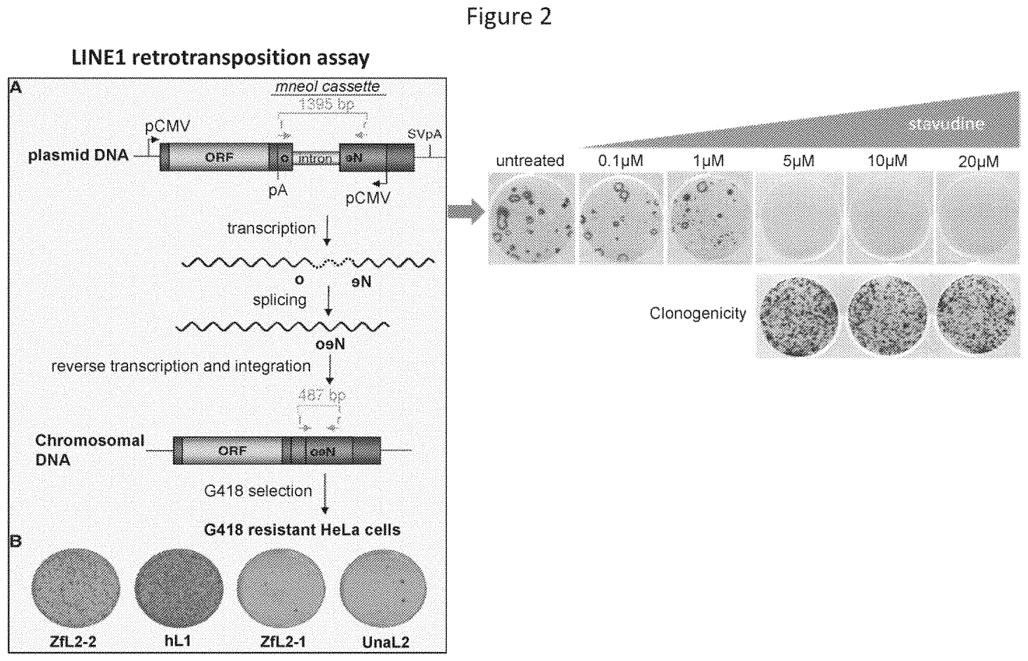

This Example is summarized in FIG. 2, which provides evidence showing the ability of a representative anti-HIV drug, HIV RT inhibitor stavudine, to inhibit retrotranspositions of LINE1 elements within non-toxic range of concentrations. Retrotransposition assay was performed according to Moran et al. Cell. 1996 87(5):917-27 (scheme shown to the left). HeLa cells were transfected with the reporter plasmid; appearance of neo-resistant colonies is indicative of retrotransposition events. Stavudine is effective at concentrations above 5 mM. Lower panel demonstrates lack of effect of the indicated concentrations of stavudine on clonogenicity of HeLa cells. It is accordingly expected that other RT inhibitors will have similar utility.

### Example 3

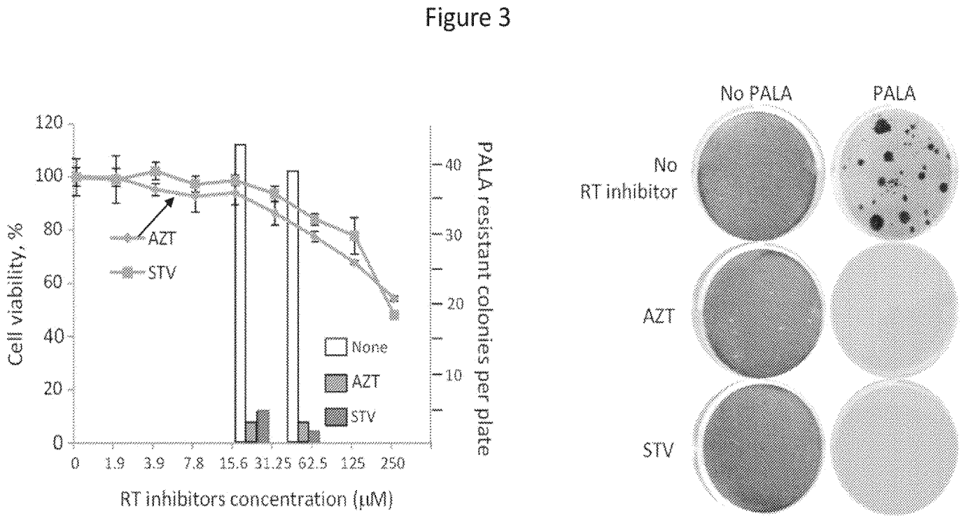

This Example is summarized in FIG. 3, which shows that representative RT inhibitors, stavudine (STV) and azidothymidine (AZT), which are demonstrated herein to be effective against LINE1 RT (see FIG. 2), suppress acquisition of drug resistant cell variants in the population of colorectal cancer cells HCT116. In particular, as a model of in vitro acquired drug resistance, we used selection of cells in the presence of N-(phosphonoacetyl)-L-aspartate (PALA), a drug which specifically inhibits the aspartate transcarbamylase activity of the multifunctional CAD enzyme and selects for amplification of the CAD gene (Stark, G. R., and Wahl, G. M. 1984. Annu. Rev. Biochem. 53: 447-503). Otto et al. (J Biol Chem. 19889. 264: 3390-3396) demonstrated a striking parallel between the ability of cell lines to become resistant to PALA and the ability of these same cells to form tumors after injection into syngeneic animals. Growth of cells in the presence of indicated concentrations of STV and AZT dramatically reduces the number of PALA resistant colonies (bar graphs in the left panel) suggesting that LINE1 RT inhibition is capable of suppressing acquisition of drug resistance and reduction of most tumorigenic tumor-initiating cells in tumor cell populations. Right panel illustrates the data shown in the graph as images of tissue culture plates treated and untreated with RT inhibitors in the presence and in the absence of PALA. Sensitivity of HCT116 cells to RT inhibitors alone were determined using quantitation of adherent cells following 72 hours of incubation in the presence of a range of concentrations of RT inhibitors (curves in the graph with the scale of cell viability shown on the right).

### Example 4

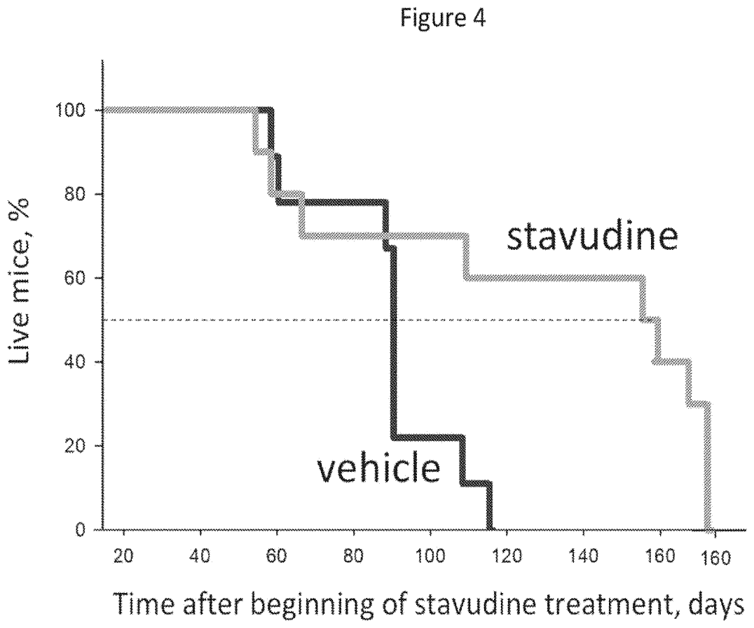

This Example is summarized in FIG. 4, which demonstrates that the HIV RT inhibitor stavudine, which is demonstrated herein to be capable of inhibiting LINE1 RT (FIG. 2), prolongs tumor-free survival of p53-null C57BL6/j male mice. p53-null mice were used as a model of reduced longevity due to spontaneous development of tumors. Mice, starting from 10 weeks of age, were kept on non-acidified drinking water with or without 1.5 mg/ml stavudine (“stavudine” and “vehicle” groups, respectively) and spontaneous deaths (or euthanasia of moribund animals or animals with tumor volumes exceeding allowed limits) were recorded. Stavudine in water extended median longevity 43% illustrating potential anti-cancer and anti-aging activity of RT inhibitor. Non-acidified water was used to preserve stability of stavudine in solution.

### Example 5

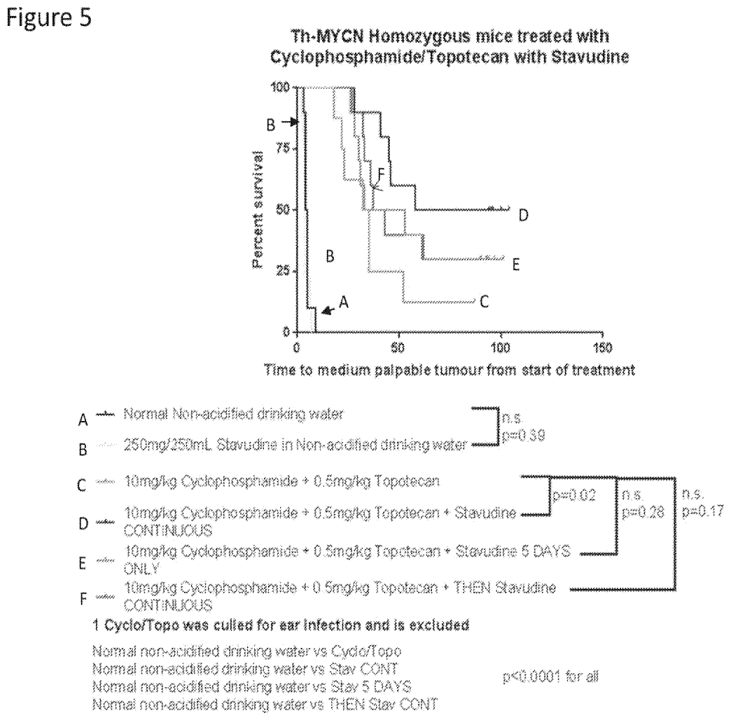

This Example is summarized in FIG. 5, which demonstrates that the representative RT inhibitor stavudine delays occurrence and reduces incidence of tumor relapses following initial response to standard chemotherapy in mouse spontaneous neuroblastoma model. In particular, 100% of Th-MYCN transgenic mice (strain 129X1/SvJ-Tg(TH-MYCN)41Waw/Nci) homozygous for MYCN transgene develop spontaneous neuroblastomas in their abdomens around 30 days of age. Mice carrying this transgene show many of the features of the human childhood disease including sensitivity to conventional treatments. Indicated treatments were started at the times of first appearance of palpable tumors. Mice were kept on non-acidified drinking water, with or without 1.5 mg/ml of stavudine, either continuously or during indicated times. As evident from the graphs, stavudine alone had no detectable antitumor effect. Treatment with combination of cyclophosphamide and topotecan prolonged tumor-free life of mice for, on average, 4 weeks followed by tumor regrowth and animal termination due to critical tumor size. Continuous treatment with stavudine significantly delayed tumor relapses extending average tumor-free survival nearly two-fold and significantly increased the proportion of disease free animals with no relapses detected within >100 days of observation. These results support prospective use of RT inhibitors as suppressors of tumor progression in combination with standard anticancer regimens.

Although the embodiments have been described in detail for the purposes of illustration, it is understood that such detail is solely for that purpose, and variations can be made therein by those skilled in the art without departing from the spirit and scope of the disclosure.

What is claimed is:

1. A method for reducing genetic instability in a population of eukaryotic cells, the method comprising introducing into the cells a reverse transcriptase (RT) inhibitor such that the activity of an RT encoded by ORF2 of LINE1 (wherein the RT is referred to as RT^{LINE}) in the cells is inhibited, and whereby the cells exhibit reduced genetic instability.

2. The method of claim 1, wherein the population of cells comprises cancer cells, pre-cancerous cells, somatic cells, or a combination thereof.

3. The method of claim 2, comprising introducing the RT inhibitor into progeny of the population of the cells, and wherein progeny of the population of cells comprise the reduced genetic instability.

4. The method of claim 2, wherein the reduced genetic instability comprises the cells exhibiting at least one of: fewer point mutations, fewer chromosomal insertions, fewer chromosomal deletions, fewer amplifications, a reduction in metastatic capability, a reduction in immortalization capacity, or a reduction in development to resistance to one or more pharmaceutical agents, wherein the reduction in genetic instability is relative to a control.

5. The method of claim 4, wherein: i) the point mutations are caused at least in in part by endonuclease activity of an integrase component of the RT; ii) the insertions comprise integration of new copies of one or more repeat elements, or integration of new pseudogenes, or a combination thereof; iii) the deletions comprise loss of a segment of a chromosome that is surrounded by integrated copies of repeat elements; iv) the amplifications comprise new copies of genome fragments comprising one or more functional genes surrounded by newly integrated copies of repeat elements.

6. The method claim 4, wherein at least some cells in the population of cells and/or progeny of said cells exhibit at least one of: i) less resistance to a pharmaceutical agent, and/or ii) reduced metastatic capability, or iii) less evidence of aging, relative to a control.

7. The method of claim 4, wherein the control comprises a value for genetic instability obtained from cells in which activity of the RT is not inhibited.

8. The method of any one of claims 1-7, wherein the population of cells are in vitro, or are present in a multi-cellular organism.

9. The method of claim 8, wherein the population of cells are mammalian cells.

10. The method of claim 9, wherein the mammalian cells are human cells.

11. The method of claim 9, wherein the population of cells is not infected by a human immunodeficiency virus (HIV) and wherein the cells in the population do not comprise an integrated HIV provirus.

12. The method of claim 11, wherein the population of cells are comprised by a solid tumor, or wherein the population of cells comprise blood cancer cells.

13. The method claim 11, wherein the population of cells comprise cancer cells in an individual, and wherein the introducing the RT inhibitor is performed over a period of time during which: i) the cancer cells do not develop resistance to a chemotherapeutic agent that is also administered to the individual during the period of time, and/or ii) the cancer cells develop less resistance, relative to a control, to a chemotherapeutic agent that is also administered to the individual during the period of time.

14. The method of claim 11, wherein the population of cells comprise somatic cells in an individual, and wherein the introducing the RT inhibitor is performed over a period of time during which a frailty index of the individual is improved, or a worsening of a frailty index of the individual is slowed relative to a control.

15. A method for characterizing whether or not a test agent(s) is a candidate for use as a reverse transcriptase (RT) inhibitor in a method of claim 11, the method comprising:
i) contacting cells that express an RT encoded by ORF2 of LINE1 (RT^{LINE}) with one or more test agents;
ii) testing for a change genetic instability in the cells; and
iii) determining that a test agent is a candidate by determining reduced genetic instability in the cells and/or their progeny relative to a control; or
iii) determining that the test agent is not a candidate by determining no reduction, or an increase, in genetic instability in the cells and/or their progeny, relative to a control.

16. The method of claim 15, wherein the reduction in genetic instability comprises the cells exhibiting at least one of: fewer point mutations, fewer chromosomal insertions, fewer chromosomal deletions, fewer amplifications, a reduction in metastatic capability, a reduction in immortalization capacity, or a reduction in development to resistance to one or more pharmaceutical agents, relative to a control.

17. The method of claim 16, wherein the method further comprises contacting the cells with a chemotherapeutic agent to determine whether or not the RT inhibitor inhibits development of resistance to the chemotherapeutic agent.

18. The method of claim 16, wherein the change in genetic instability is determined using a detectable reporter, wherein the detectable reporter is indicative of a DNA damage response.

19. A method of reducing cells that exhibit genetic instability comprising administering to an individual in need thereof a composition comprising one or more agents that are capable of selectively killing the cells that exhibit the genetic instability, wherein the cells that exhibit the genetic instability are identified by expression of functional LINE1.

20. The method of claim 19, wherein the one or more agents comprise all or a segment of one or two proteins encoded by LINE1 elements (ORF1 and ORF2), or the one or more agents comprise an expression vector(s) encoding the one or two proteins or the segment(s) thereof, wherein the protein(s) or the segment(s) thereof can stimulate an immune response against the cells that exhibit the genetic instability.

21. The method of claim 20, wherein the composition comprising the one or more agents further comprises one or more immunoadjuvants and/or immunomodulators, and/or the expression vector(s) encode the one or more immunoadjuvants and/or immunomodulators.

22. The method of any one of claims 19-21, wherein the one or more agents comprise nucleoside analog reverse-transcriptase inhibitors or nucleotide analog reverse-transcriptase inhibitors.

23. The method of claim 22, wherein the one or more agents comprise stavudine or lamivudine or a combination thereof.

24. A method of identifying a stimulus that generates genetic instability comprising exposing cells to a test stimulus and testing the cells for an increase in activity of an RT encoded by ORF2 of LINE1 (RT^{LINE}), wherein an increase in in the activity of the RT indicates the stimulus generates genetic instability.

25. A method for identifying an individual as a candidate for treatment with an inhibitor of reverse transcriptase (RT) encoded by ORF2 of LINE1 (RT^{LINE}) or with a vaccine that stimulates expression of an immune response against cells that exhibit a genetic instability that can be identified by expression of functional LINE1, the method comprising testing a sample from the individual to determine a measure of genetic instability and comparing the measure of the genetic instability to a control, wherein more genetic instability relative to the control indicates the individual is a candidate for the treatment.

26. The method of claim 25, wherein the measure of genetic instability comprises at least one value for: point mutations, chromosomal insertions, chromosomal deletions, gene amplifications, metastatic capability of cells from the individual, immortalization capacity of cells from the individual, or expression of LINE1 DNA repeats in cells from the individual.

27. The method of claim 25 or claim 26, further comprising administering the inhibitor of the RT and/or the vaccine to the individual.

28. A method for monitoring the efficacy of a treatment for inhibiting development of resistance to treatment with a pharmaceutical agent, the method comprising testing a sample of cells from the individual for a measure of genetic instability at a first time point during the treatment and comparing the measure to a control, wherein a lack of increase in the genetic instability relative to the control indicates adequate efficacy of the treatment.

29. The method of claim 28, wherein an increase in the genetic instability at the first time point indicates inadequate efficacy of the treatment.

30. The method of claim 29, comprising adjusting the dosage of the treatment and testing a second sample of cells from the individual for the measure of the genetic instability to determine if the dosage adjustment was adequate to inhibit development of the resistance.

31. The method of any one of claims 28-30, wherein the treatment is a cancer treatment, and wherein the agent is a chemotherapeutic agent or a vaccine.

32. A method for prophylaxis and/or therapy for a pathology correlated with accumulation of somatic cells capable of spontaneously generating genetic alterations independently of cell divisions, wherein such cells can be characterized by expression of functional LINE1 elements, the method comprising administering to the individual a reverse transcriptase (RT) inhibitor such that the activity of an RT encoded by ORF2 of LINE1 (RT^{LINE}) in the somatic cells is inhibited, and/or administering to the individual a vaccine such that an immunological response to the somatic cells is stimulated.

33. The method of claim 32, wherein the pathology comprises cancer a proliferation disorders selected from: myelodysplastic syndrome or benign tumors, or aberrant immunomodulation that is induction and/or exacerbation of inflammation that initiates or accelerates aging and/or malignant transformation of the somatic cells.

34. The method of claim 33, wherein the pathology comprises spontaneous aging, and/or one or more conditions associated with aging and/or spontaneous aging.

35. The method of any one of claims 32-34, wherein a change in the pathology can be detected by determining a change in frailty index for the individual.

36. The method of claim 35, wherein the change in the frailty index is measured at distinct time points to evaluate efficacy of the method of prophylaxis and/or therapy.

37. A method for treating and/or preventing age-related conditions in an individual comprising administering to an individual in need thereof a composition comprising one or more agents that are capable of selectively killing the cells that exhibit genetic instability, wherein the cells that exhibit the genetic instability can be identified by expression of functional LINE 1.

38. The method of claim 37, wherein the one or more agents comprise all or a segment of one or two proteins encoded by LINE1 elements (ORF1 and ORF2), or the one or more agents comprise an expression vector(s) encoding the one or two proteins or the segment(s) thereof, wherein the protein(s) or the segment(s) thereof can stimulate an immune response against the cells that exhibit the genetic instability.

39. The method of claim 37, wherein the composition comprising the one or more agents further comprises one or more immunoadjuvants and/or immunomodulators, and/or the expression vector(s) encode the one or more immunoadjuvants and/or immunomodulators.

40. The method of any one of claims 37-39, wherein the one or more agents comprise Nucleoside analog reverse-transcriptase inhibitors or Nucleotide analog reverse-transcriptase inhibitors.

41. A method of sensitizing cancer cells to a chemotherapeutic agent, the method comprising introducing into the cancer cells the chemotherapeutic agent and a reverse transcriptase (RT) inhibitor, wherein the cancer cells are not infected by a human immunodeficiency virus (HIV) and wherein the cancer cells do not comprise an integrated HIV provirus.
